(19) United States
(12) Patent Application Publication
(10) Pub. No.: US 2019/0150777 A1
(43) Pub. Date: May 23, 2019

(54) DUAL-SUPPLY ANALOG CIRCUITRY FOR SENSING SURFACE EMG SIGNALS

(71) Applicant: CTRL-labs Corporation, New York, NY (US)

(72) Inventors: Ning Guo, Brooklyn, NY (US); Jonathan Caza Reid, Brooklyn, NY (US)

(73) Assignee: CTRL-labs Corporation, New York, NY (US)

(21) Appl. No.: 15/816,435

(22) Filed: Nov. 17, 2017

(51) Int. Cl. A61B 5/0492; H03F 3/45; A61B 5/04; A61B 5/00

(52) U.S. Cl. CPC A61B 5/0492; H03F 3/45475; A61B 5/04012; A61B 2562/043; H03F 2203/45022; H03F 2203/45138; A61B 2562/0209; A61B 5/6824

(57) ABSTRACT

Dual-supply analog circuitry for amplifying surface EMG (sEMG) signals is described. The circuitry includes a differential amplifier configured to be powered from dual-supply voltages. A positive input terminal of the differential amplifier is configured to be DC-coupled to a first sEMG electrode of a dry sEMG electrode pair and a negative input terminal of the differential amplifier is configured to be DC-coupled to a second sEMG electrode of the dry sEMG electrode pair.

### BACKGROUND

High-quality surface electromyography (sEMG) signals are typically acquired from wet electrodes in a laboratory setting using skin preparations that require application of a gel or paste at the electrode-skin interface to improve the conductivity between the skin and the electrodes. Data acquisition circuitry for sEMG recordings typically include an analog front-end amplifier design configured as an AC-coupled (e.g., capacitively-coupled) input stage to remove the DC offset voltage originating at the electrode-skin interface prior to amplification of the sEMG signals. The AC-coupled input stage amplifier is often powered by a single-supply voltage referenced to ground, and the input stage is typically biased up to the midpoint voltage of the amplifier to achieve maximum input dynamic range. The biasing is achieved by including resistors at an input stage of the amplification circuitry, where the resistors typically have values much lower than the input impedance of the amplifier.

### SUMMARY

Some embodiments are directed to a sEMG system. The sEMG system comprises a pair of dry sEMG electrodes, and amplification circuitry comprising a first differential amplifier configured to be powered from dual-supply voltages, wherein a first sEMG electrode of the pair is DC-coupled to a positive input terminal of the first differential amplifier and a second sEMG electrode of the pair is DC-coupled to a negative input terminal of the first differential amplifier.

Some embodiments are directed to amplification circuitry. The amplification circuitry comprises a first differential amplifier configured to be powered by dual-supply voltages, wherein the first differential amplifier is further configured to have a common-mode voltage of approximately 0 volts, wherein an input impedance of the first differential amplifier is at least 1 Giga Ohm, and wherein a gain of the first differential amplifier is less than 15.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale.

FIG. 1 is a schematic diagram of components of a sEMG system in accordance with some embodiments of the technology described herein;

FIG. 2 illustrates a wristband having sEMG sensors arranged circumferentially thereon, in accordance with some embodiments of the technology described herein;

FIG. 3 illustrates a user wearing the wristband of FIG. 2 while typing on a keyboard, in accordance with some embodiments of the technology described herein;

FIG. 4 illustrates AC-coupled amplification circuitry that may be used to amplify sEMG signals;

FIG. 5 illustrates single stage dual-supply DC-coupled amplification circuitry in accordance with some embodiments of the technology described herein;

FIG. 6 illustrates multi-stage DC-coupled amplification circuitry in accordance with some embodiments of the technology described herein;

FIG. 7 illustrates a sEMG waveform recorded using a sEMG system designed in accordance with some embodiments of the technology described herein;

FIG. 8 illustrates a zoomed-in portion of the sEMG waveform of FIG. 7; and

FIG. 9 is a schematic diagram of components of a sEMG system that includes at least one galvanic isolation component in accordance with some embodiments of the technology described herein.

### DETAILED DESCRIPTION

Obtaining consistent high-quality sEMG signals using sEMG electrodes and conventional signal processing techniques is challenging, in part due to impedance mismatches at the interface between skin and the electrodes. For applications that require near real-time analysis of sEMG signals, the acquisition of consistent high-quality signals is important to be able to iterate quickly on the recorded data.

Some conventional techniques for addressing impedance mismatches at the electrode-skin interface in laboratory and clinical settings include the use of wet electrodes or the use of dry electrodes in combination with skin preparations (e.g., shaving, sanding, hydrating with cream). Even when used with skin preparations, dry electrodes tend to have considerable variability in the impedance caused by electrode-skin environment variations and these mismatches result in the severe degradation in the common-mode rejection ratio of amplifiers to which the sEMG signals are provided for amplification. The inventors have appreciated that conventional techniques used in laboratory or clinical settings for acquiring high-quality sEMG signals are not desirable or feasible for consumer applications, in which users may not want to apply gels/creams for wet electrodes or perform skin preparations for dry electrodes. To this end, some embodiments are directed to techniques for mitigating impedance mismatches at the electrode-skin interface of dry sEMG electrodes that produce high-quality sEMG signals without the use of skin preparations. Additionally, some embodiments are directed to techniques for improving the robustness of a wearable sEMG device and improving the consistency of recorded sEMG data.

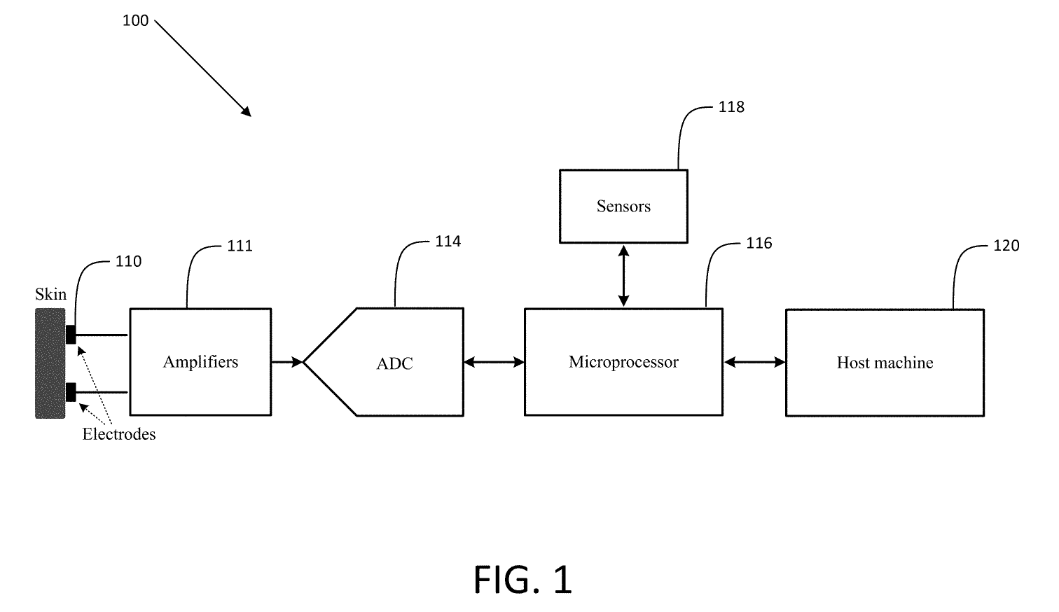

FIG. 1 schematically illustrates components of a sEMG system 100 in accordance with some embodiments. System 100 includes a pair of dry sEMG electrodes 110. In some embodiments, electrodes 110 may be arranged as a portion of a wearable device configured to be worn on or around part of a user's body. For example, in one non-limiting example, a plurality of sEMG sensors including sEMG electrodes (e.g., electrodes 110) are arranged circumferentially around an adjustable and/or elastic band such as a wristband or armband configured to be worn around a user's wrist or arm. Alternatively, at least some of the sEMG sensors may be arranged on a wearable patch configured to be affixed to a portion of the user's body.

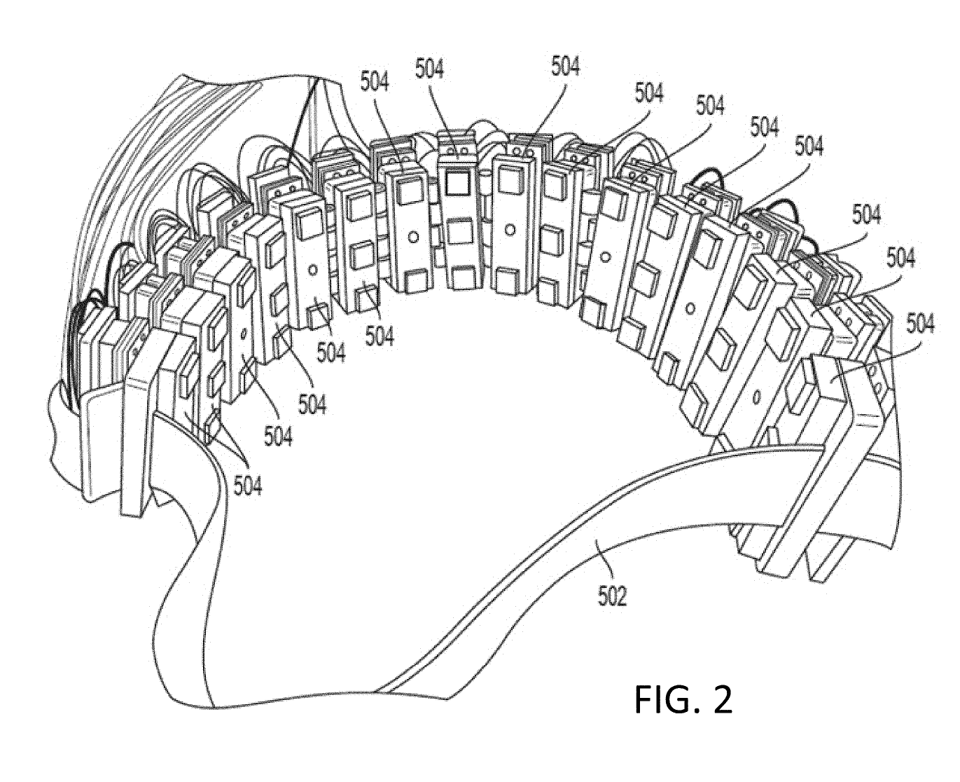

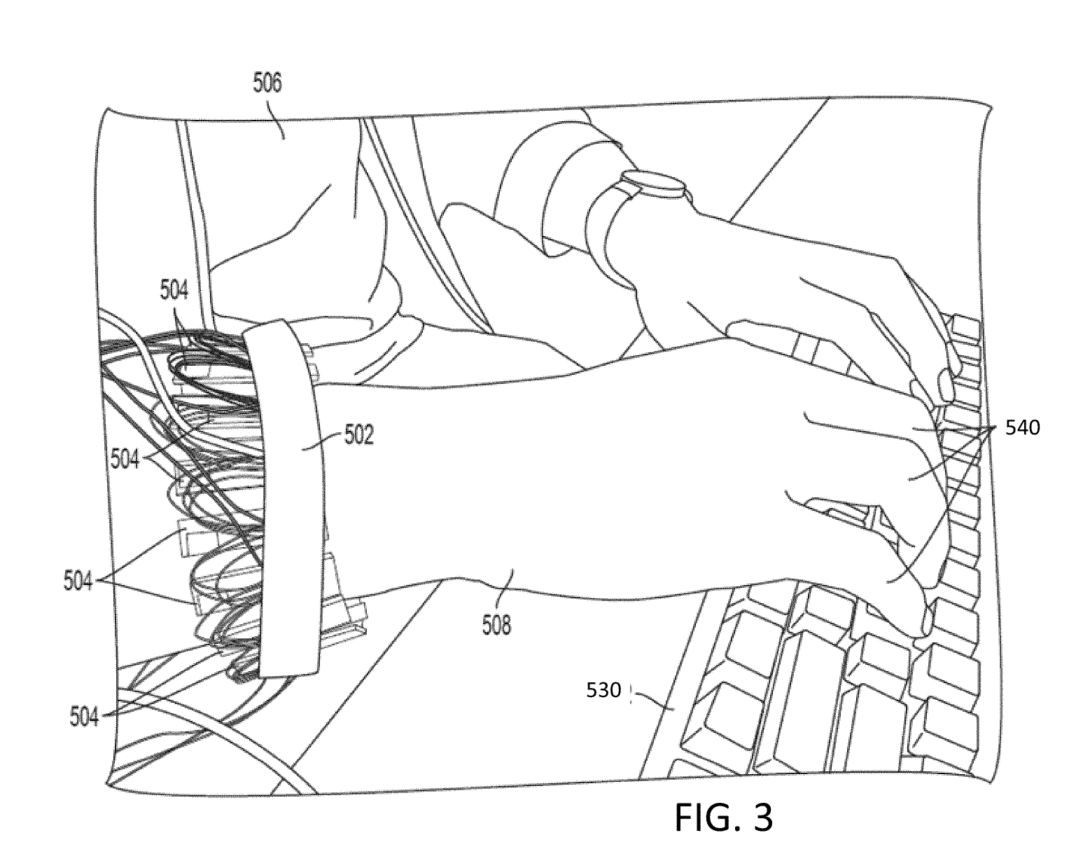

In one implementation, sixteen sEMG sensors including dry sEMG electrodes are arranged circumferentially around an elastic band configured to be worn around a user's lower arm. For example, FIG. 2 shows sEMG sensors 504 arranged circumferentially around elastic band 502. It should be appreciated that any suitable number of sEMG sensors having any suitable number of dry sEMG electrodes may be used and the number and arrangement of sensors/electrodes may depend on the particular application for which the wearable device is used. For example, as shown in FIG. 2, some of the sEMG sensors 504 include two dry sEMG electrodes, whereas other of the sEMG sensors 504 include three dry sEMG electrodes, with the middle of the three electrodes being a ground electrode. The ground electrode may be included on one or more of the sEMG sensors 504 to, for example, further bias the skin potential and/or to filter out noise. Although the schematic diagrams in FIGS. 1, 4-6 and 9 illustrate only two electrodes being connected to amplifier 111/112/113, it should be appreciated that for sEMG sensors 504 in which three (or more) electrodes are used, a corresponding number of connections between the electrodes and the amplification circuitry would be included. In one example application of the technology described herein, FIG. 3 shows a user 506 wearing elastic band 502 on hand 508. In this way, sEMG sensors 504 may be configured to record sEMG signals as a user controls keyboard 530 using fingers 540.

Surface potentials recorded by sEMG electrodes are typically small and amplification of the signals recorded by the sEMG electrodes is typically desired. As shown in FIG. 1, dry sEMG electrodes 110 are coupled to amplification circuitry 111, configured to amplify the sEMG signals recorded by the electrodes. The output of the amplification circuitry 111 is provided to analog-to-digital converter (ADC) circuitry 114, which converts the amplified sEMG signals to digital signals for further processing by microprocessor 116. Microprocessor 116 may be implemented by one or more hardware processors. The processed signals output from microprocessor 116 may be interpreted by host machine 120, examples of which include, but are not limited to, a desktop computer, a laptop computer, a smartwatch, a smartphone, or any other computing device. In some implementations, host machine 120 may be configured to output one or more control signals for controlling a physical or virtual device based, at least in part, on an analysis of the signals output from microprocessor 116.

As shown, sEMG system 100 also includes sensors 118, which may be configured to record types of information about a state of a user other than sEMG information. For example, sensors 118 may include, but are not limited to, temperature sensors configured to measure skin/electrode temperature, inertial measurement unit (IMU) sensors configured to measure movement information such as rotation and acceleration, humidity sensors, heart-rate monitor sensors, and other bio-chemical sensors configured to provide information about the user and/or the user's environment.

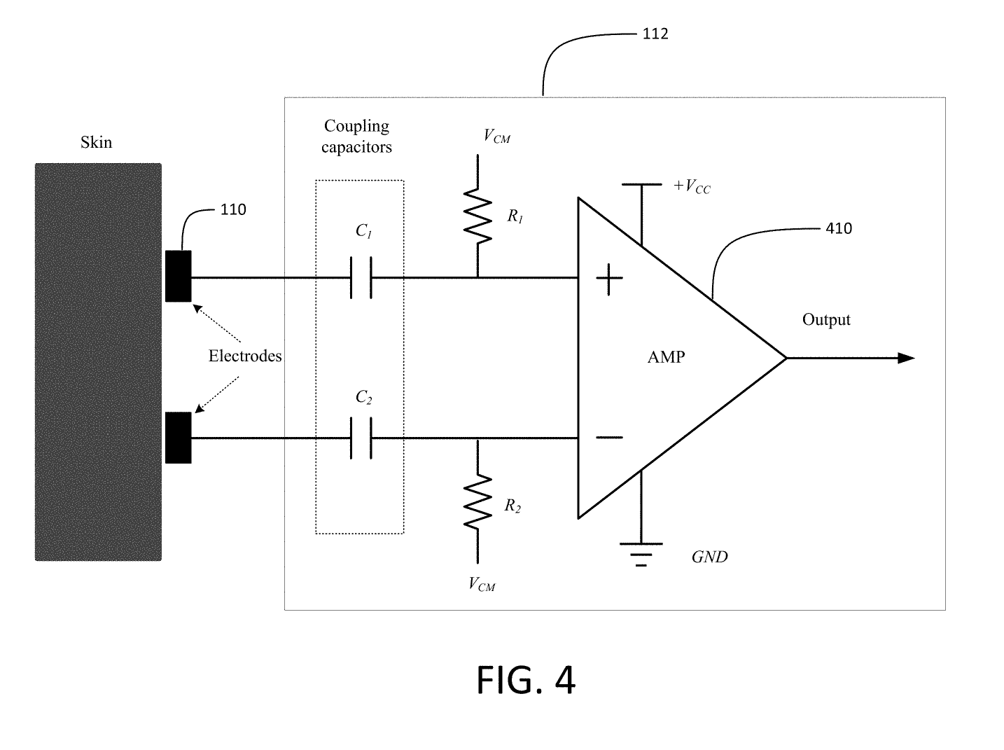

An implementation of amplification circuitry 111 shown in FIG. 1 is illustrated in FIG. 4 as amplification circuitry 112. Amplification circuitry 112 may be used in some conventional sEMG data acquisition circuitry. As shown, differential amplifier 410 is AC-coupled (e.g., capacitively-coupled) to electrodes 110 via coupling capacitors C₁ and C₂. Capacitors C₁ and C₂ remove the DC offset voltage originating at the electrode-skin interface prior to amplification by amplifier 410. Amplifier 410 is powered using a single supply (e.g., a battery) voltage +V_{CC} referenced to ground. Such an implementation exhibits significant power line noise (e.g., 50 Hz/60 Hz) coupling, introducing noise in the recorded signals. As discussed briefly above, a best practice to achieve maximum input dynamic range for differential amplifiers is to bias the amplifier to the midpoint voltage of amplifier 410 (e.g., +V_{CC}/2 in the amplification circuitry of FIG. 4). To bias the amplifier's inputs, amplification circuitry 112 shown in FIG. 4 also includes resistors R₁ and R₂, connected between inputs of amplifier 410 and a voltage V_{CM}. Resistors R₁ and R₂ typically have values (e.g., tens of Mega Ohms) much lower than the input impedance of amplifier 410, thus lowering the total input impedance of amplification circuitry 112. Due to its relatively low input impedance, amplification circuitry 112 is particularly susceptible to impedance mismatches at the electrode-skin interface.

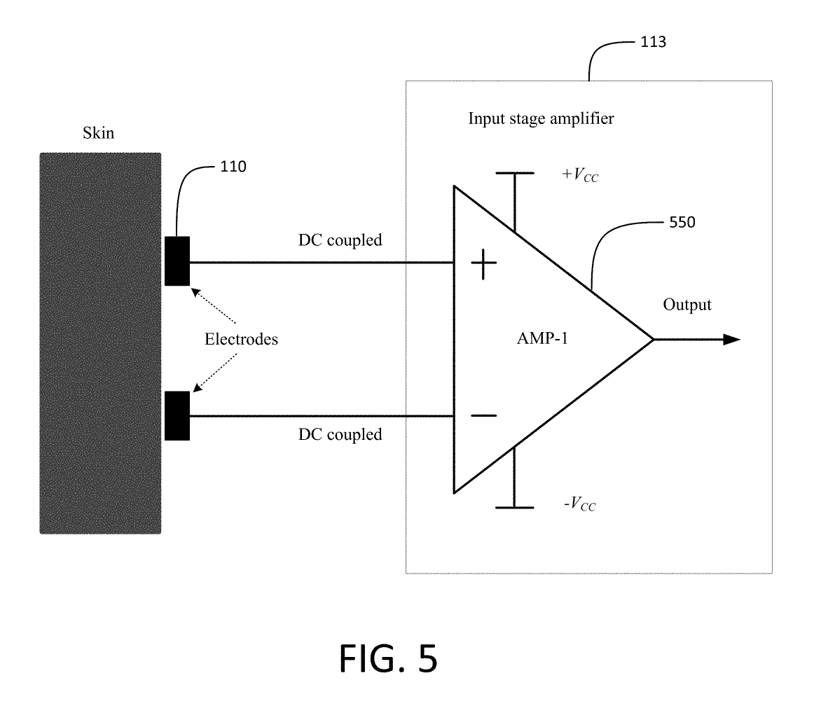

An implementation of amplification circuitry 111 shown in FIG. 1 is illustrated in FIG. 5 as amplification circuitry 113. Amplification circuitry 113 is configured to at least partially mitigate the susceptibility of the amplification circuitry to electrode-skin interface impedance mismatches in accordance with some embodiments. As discussed in more detail below, some embodiments are directed to amplification circuitry configured to reduce impedance mismatches at the electrode-skin interface and/or reduce the effect of impedance mismatches at the electrode-skin interface using a selection of amplifier characteristics and/or setting the midpoint voltage of the amplifier inputs to a potential of the human body/skin (e.g., approximately 0 volts).

Amplification circuitry 113 shown in FIG. 5 includes differential amplifier 550 configured to be DC-coupled (e.g., resistively-coupled) to dry sEMG electrodes 110. Any suitable type of dry sEMG electrodes (e.g., dry sEMG electrodes have any shape or size) may be used with embodiments of the amplification circuitry described herein. Unlike amplifier 410, which is capacitively-coupled to the dry sEMG electrodes, DC coupling the inputs of amplifier 550 to electrodes 110 does not remove a DC offset voltage prior to amplification. Rather than removing a DC offset, differential amplifier 550 is configured to be powered by dual voltage sources (shown as +V_{CC} and −V_{CC}), which allows the common-mode voltage of the amplifier 550 to be matched to the voltage potential level of the human body/skin (e.g., typically about 0V). Any suitable values of +V_{CC} and −V_{CC} may be used, and embodiments are not limited in this respect. By matching the common-mode voltage of the amplifier 550 to the voltage potential level of the human body/skin using a dual-supply power scheme, removal of DC offset introduced at the electrode-skin interface prior to amplification is not necessary. As such, with the gain chosen appropriately, amplifier 550 is able to function properly with DC coupling to the electrodes 110 without saturating the output of the amplifier.

Unlike amplifier 410, which has a relatively low input impedance (e.g., tens to hundreds of Mega Ohms), in some embodiments, the input impedance of amplifier 550 may be selected to be higher than would typically be used for amplifier 410. The higher input impedance of amplifier 550 protects against variability in the impedance at the electrode-skin interface. For example, in some embodiments the input impedance of amplifier 550 is at least one Giga Ohm. In some embodiments, the input impedance of amplifier 550 is at least one Tera Ohm. Additionally, the amplifier 550 may be configured to have a relatively low gain. In some embodiments, the gain of amplifier 550 is less than 100. In some embodiments, the gain of amplifier 550 is less than 50, less than 20, or less than 15. In some embodiments, the gain of amplifier 550 is approximately 10.

As should be appreciated from the foregoing discussion, amplification circuitry 113 designed in accordance with some embodiments includes at least two aspects that operate together to enable differential amplifier 550 to be DC-coupled to dry sEMG electrodes without introduction of appreciable noise at the electrode-skin interface—selection of amplifier characteristics (e.g., relatively high input impedance, relatively low gain), and use of a dual-supply voltage power scheme, which enables the common-mode voltage of the amplifier to be matched to the voltage potential of the human body/skin.

In some embodiments, one or more protection resistors (not shown) may be arranged between the inputs of amplifier 550 and the dry electrodes 110. For example, a first resistor may be arranged between a first sEMG electrode of the dry electrode pair and the positive input terminal of amplifier 550, and a second resistor may be arranged between a second sEMG electrode of the dry electrode pair and the negative input terminal of amplifier 500. Protection resistors used in accordance with some embodiments may have resistance values on the order of 100 kilo Ohms.

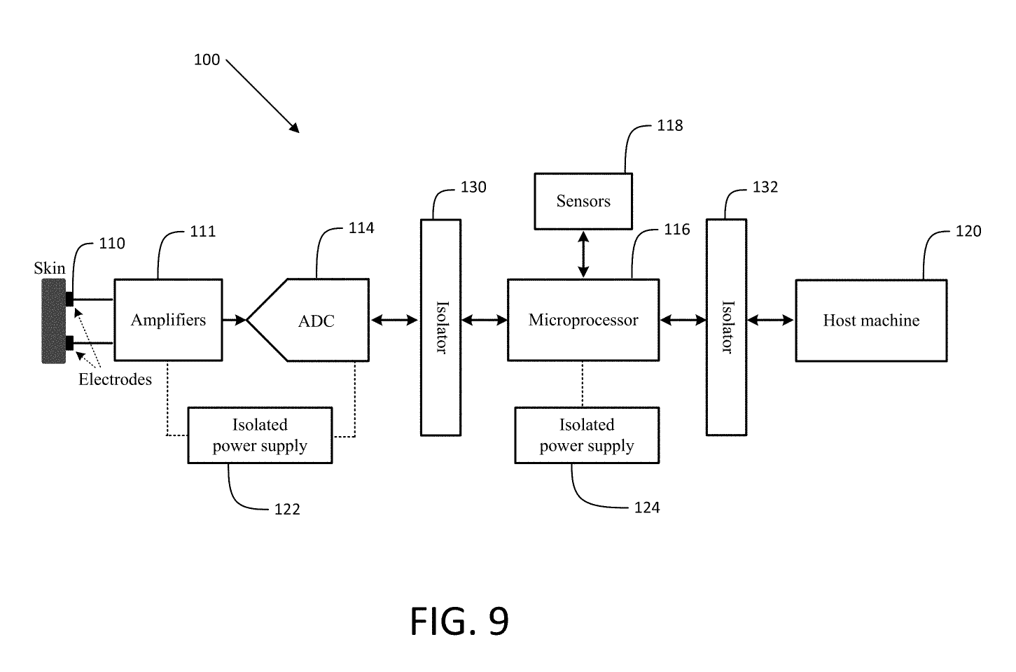

In some embodiments, isolation circuitry is used to provide further noise isolation. As shown in FIG. 9, isolator 130 is arranged between ADC 114 and microprocessor 116, and isolator 132 is arranged between microprocessor 116 and host machine 120. These isolators are configured to allow digital signals to pass through uni-directionally or bi-directionally, while providing galvanic isolation for the digital signals. Isolators 130, 132 may be implemented using any suitable isolation techniques that provide galvanic isolation. Examples of techniques for implementing isolators 130, 132 include, but are not limited to, optical coupling techniques (e.g., using optical couplers), magnetic coupling techniques (e.g., using transformers) and capacitive coupling techniques (e.g., using a capacitive isolation-barrier). In combination with isolators 130, 132, isolated power supplies 122, 124 may be used to provide operating power for each isolated part in the sEMG system 100. The isolated power supplies 122, 124 may be implemented, for example, as independent power sources such as batteries, or as power sources having power derived from another power source and including isolation such as a transformer-based DC-DC converter. For example, an isolated power source configured as a derived power source may receive power from host machine 120 (e.g., via a USB port included on host machine 120) and use transformer circuitry to transform the relatively noisy power from the host machine into power exhibiting substantially less noise.

Although sEMG system 100 shown in FIG. 9 is illustrated as having two isolators 130 and 132, it should be appreciated that a single isolator 130 or 132 may be used, and embodiments are not limited in this respect. In embodiments that include a single isolator, it should be appreciated that only a single isolated power supply may be needed to provide power to the isolated part of the sEMG system. Additionally, although two isolated power supplies 122, 124 are shown in FIG. 9, it should be appreciated that a single isolated power supply may be used in some embodiments to provide isolated power to multiple isolated components of the system. For example, a single battery may be used to provide power to one isolated part of the system, and power derived from the single battery (e.g., using transformer circuitry) may be used to power another isolated part of the system. In another example, power derived from the host computer (e.g., from a USB port) or some other source may be used to power multiple isolated parts of the system by using multiple galvanic power isolation techniques (e.g., by using multiple transformers).

Differential amplifier 550 may be implemented using any suitable type of circuit components having the characteristics described above. In some embodiments, differential amplifier 550 is implemented using a plurality of Field Effect Transistors (FETs), examples of which include, but are not limited to, metal oxide field effect transistors (MOSFETs) and junction gate field effect transistors (JFETs).

As noted above, differential amplifier 550 may be configured to have a relatively low gain. Accordingly, in some embodiments, if additional amplification of the recorded sEMG signals is desired, amplification circuitry 113 may be configured to include one or more additional amplification stages coupled to the output of differential amplifier 550.

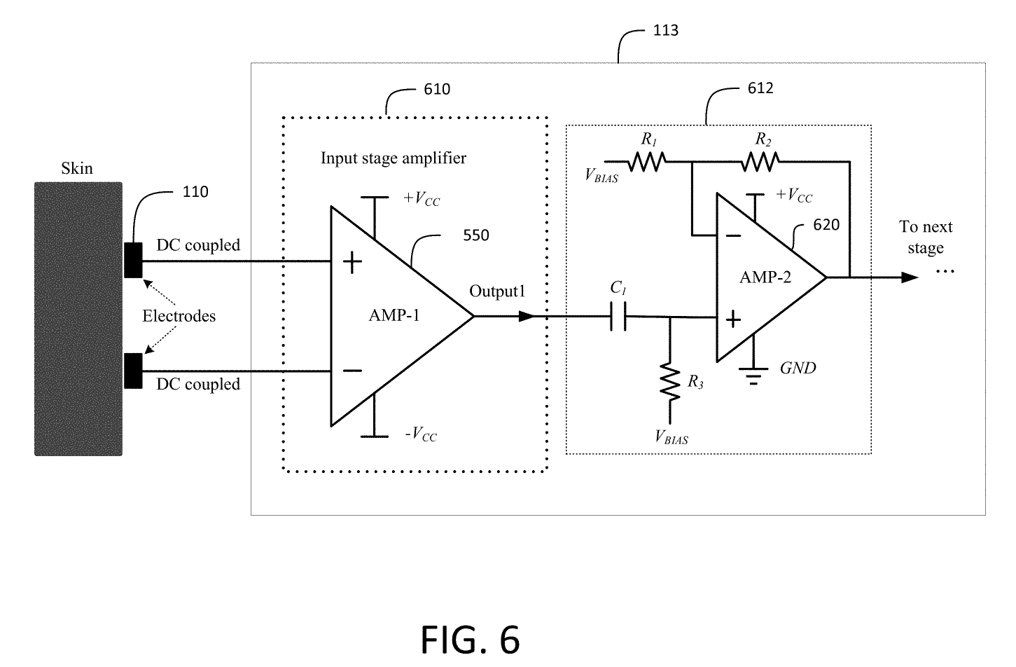

FIG. 6 illustrates an embodiment of amplification circuitry 113 including multiple amplification stages. As shown, amplification circuitry 113 includes a first amplification stage 610 and a second amplification stage 612 having an input coupled to the output of the first amplification stage 610. In the example of FIG. 6, first amplification stage 610 is configured in the same manner as described previously with regard to FIG. 5 and, for the sake of brevity, is not discussed further. Second amplification stage 612 may have the same configuration as first amplification stage 610 (e.g., powered by dual-supply voltages) or one or more aspects of the second amplification stage 612 may be different than the first amplification stage 610. As shown in the example of FIG. 6, second amplification stage 612 includes amplifier 620 having an input terminal AC-coupled via capacitor C₁ to the output of the first amplification stage 610 to remove a DC component of the signal. However, it should be appreciated that the inputs of amplifier 620 may alternatively be DC-coupled to the output of the first amplification stage 610. Additionally, amplifier 620 is shown in the example of FIG. 6 as being powered by a single-supply scheme, though it should be appreciated that amplifier 620 may alternately be powered by a dual-supply scheme, depending on requirements of the subsequent signal chain.

Amplifier 620 may be configured to have any suitable gain (e.g., as low as 1 or as high as 1000) as needed by the subsequent signal chain components. In some embodiments, the gain of amplifier 620 may be higher than the gain of amplifier 550 in the first amplification stage 610, whereas in other embodiments the gain of amplifier 620 may the same as or less than the gain of amplifier 550.

As schematically illustrated in FIG. 6, the output of the second amplification stage may be provided to one or more additional amplification stages included, but not shown, in amplification circuitry 113. Any number of amplification stages may be included in amplification circuitry 113, and embodiments are not limited in this respect. For example, some embodiments include three stages of amplification in amplification circuitry 113.

Amplification circuitry 113 may be implemented using a single-ended analog signal representation, a differential analog signal representation, or a combination of a single-ended analog signal representation and a differential analog signal representation. As schematically illustrated in FIG. 6, the output of the first amplification stage 610 (shown as “Output 1”) is a single-ended analog signal. Alternatively, amplifier 550 may be implemented using a differential-input, differential-output architecture in which the output of the first amplification stage 610 is a differential analog signal (e.g., Output1+, Output1−) and the second amplification stage 612 is designed to process the differential analog signal output from the first amplification stage 610. For embodiments that include multiple amplification stages, a single-ended analog signal representation or a differential analog signal representation may be used at the output of each amplification stage, and the subsequent amplification stage may be designed accordingly. For example, in embodiments that include three amplification stages, the first amplification stage output may be a differential analog signal, the second amplification stage output may be a differential analog signal and the third amplification stage output may be a single-ended analog signal. In some embodiments, a differential analog signal representation is used for all amplification stages.

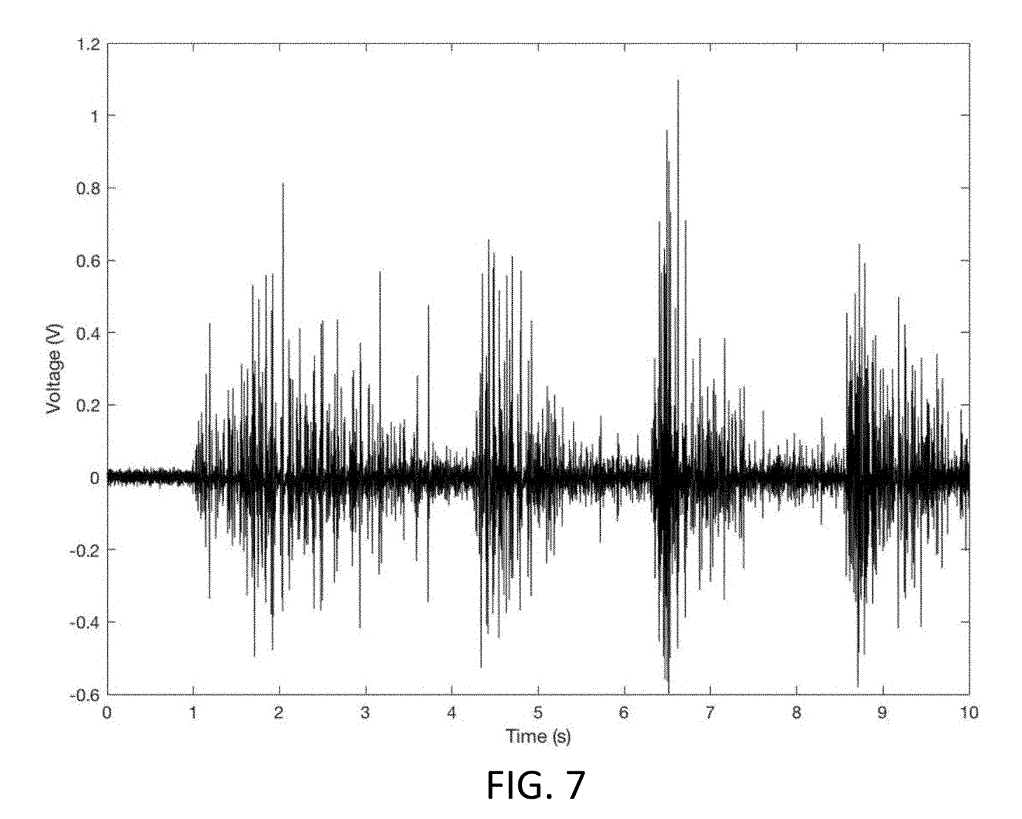

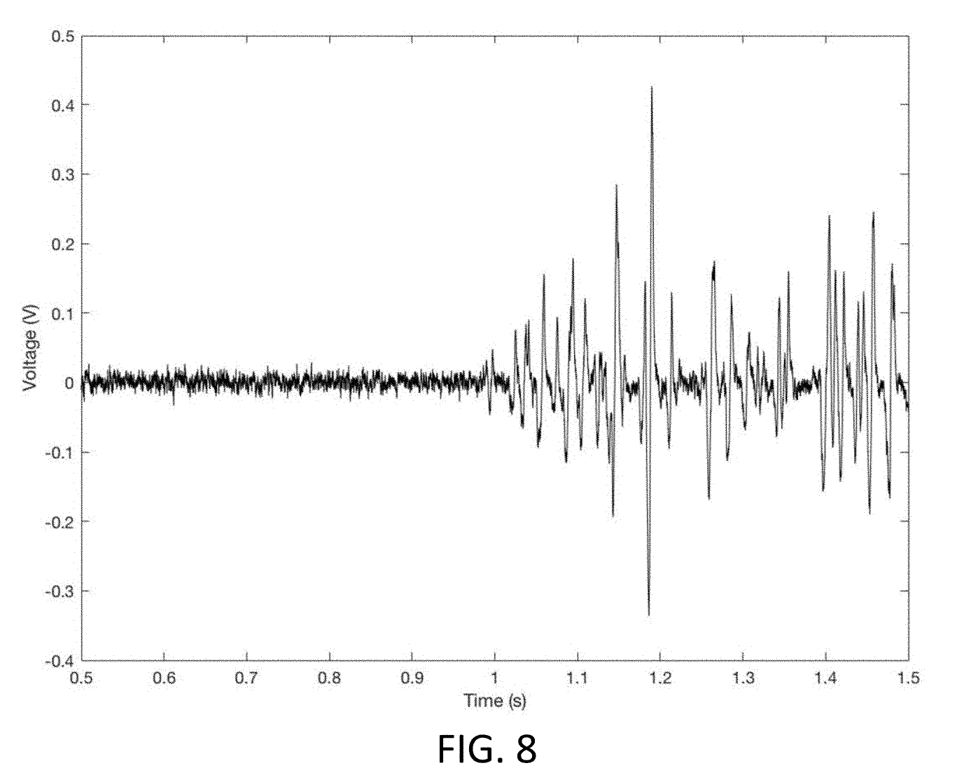

FIGS. 7 and 8 illustrate sEMG waveforms processed using amplification circuitry 113 designed in accordance with some embodiments. FIG. 7 shows sEMG signals recorded for a period of ten seconds by dry electrodes placed on unprepared skin of a user and processed by the DC-coupled dual-supply amplification circuitry described herein. FIG. 8 shows a zoomed in version of a portion of the sEMG waveform of FIG. 7 in the range from 0.5 to 1.5 seconds to show additional signal details. As can be appreciated from the waveform in FIG. 8, the sEMG signal recorded starting around 1 second is clearly distinguishable from the noise recorded on the channel between 0.5 and 1 seconds.

The implementations of DC-coupled amplification circuitry described herein employ discrete analog circuit components. However, it should be appreciated that all or portions of the amplification circuitry and/or associated circuitry in the signal chain may alternatively be implemented using one or more application specific integrated circuits (ASICs) or using any other custom silicon implementation, and embodiments are not limited in this respect.

Various aspects of the apparatus and techniques described herein may be used alone, in combination, or in a variety of arrangements not specifically discussed in the embodiments described in the foregoing description and is therefore not limited in its application to the details and arrangement of components set forth in the foregoing description or illustrated in the drawings. For example, aspects described in one embodiment may be combined in any manner with aspects described in other embodiments.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A surface electromyography (sEMG) system comprising:
a pair of dry sEMG electrodes; and
amplification circuitry comprising a first differential amplifier configured to be powered from dual-supply voltages,
wherein a first sEMG electrode of the pair of dry sEMG electrodes is DC-coupled to a positive input terminal of the first differential amplifier and a second sEMG electrode of the pair of dry sEMG electrodes is DC-coupled to a negative input terminal of the first differential amplifier.

2. The sEMG system of claim 1, wherein the first differential amplifier is configured to have a common-mode voltage of approximately 0 volts.

3. The sEMG system of claim 1, wherein the first differential amplifier is configured to have an input impedance of at least one Giga Ohm.

4. The sEMG system of claim 3, wherein the first differential amplifier is configured to have an input impedance of at least one Tera Ohm.

5. The sEMG system of claim 1, wherein the first differential amplifier is configured to have a gain of less than 50.

6. The sEMG system of claim 5, wherein the first differential amplifier is configured to have a gain of less than 15.

7. The sEMG system of claim 1, wherein the first differential amplifier comprises a field-effect transistor (FET).

8. The sEMG system of claim 1, further comprising:
a first resistor arranged between the first sEMG electrode and the positive input terminal of the first differential amplifier; and
a second resistor arranged between the second sEMG electrode and the negative input terminal of the first differential amplifier.

9. The sEMG system of claim 1, wherein the amplification circuitry further comprises:
a second differential amplifier having an input coupled to an output terminal of the first differential amplifier.

10. The sEMG system of claim 9, wherein the second differential amplifier is configured to be powered from a single supply voltage.

11. The sEMG system of claim 9, wherein the second differential amplifier is configured to be powered from dual-supply voltages.

12. The sEMG system of claim 9, wherein the second differential amplifier is AC-coupled to the output terminal of the first differential amplifier.

13. The sEMG system of claim 9, wherein a gain of the second differential amplifier is larger than a gain of the first differential amplifier.

14. The sEMG system of claim 9, wherein the amplification circuitry further comprises:
a third differential amplifier having an input coupled to an output of the second differential amplifier.

15. The sEMG system of claim 1, further comprising:
an analog-to-digital converter coupled to an output of the amplification circuitry; and
at least one processor coupled to the analog-to-digital converter, wherein the at least one processor is configured to perform digital signal processing on a signal received from the analog-to-digital converter.

16. The sEMG system of claim 1, wherein the pair of dry sEMG electrodes are arranged on a wearable device configured to be worn on or around a body part of the user.

17. The sEMG system of claim 1, further comprising:
at least one isolation component configured to provide galvanic isolation between components of the sEMG system having digital data communication; and
at least one isolated power supply configured to provide operating power to one or more of the components of the sEMG system isolated using the at least one isolation component.

18. Amplification circuitry, comprising:
a first differential amplifier configured to be powered by dual-supply voltages, wherein the first differential amplifier is further configured to have a common-mode voltage of approximately 0 volts, wherein an input impedance of the first differential amplifier is at least 1 Giga Ohm, and wherein a gain of the first differential amplifier is less than 15.

19. The amplification circuitry of claim 16, further comprising:
a second differential amplifier having an input coupled to an output terminal of the first differential amplifier.

20. The amplification circuitry of claim 19, wherein the second differential amplifier is configured to be powered by dual-supply voltages.
